(19) United States
(12) Patent Application Publication
(10) Pub. No.: US 2014/0363673 A1
(43) Pub. Date: Dec. 11, 2014

(54) THERAPEUTIC AGENT FOR INFLAMMATORY BOWEL DISEASE

(71) Applicants: Saburo Minami, Tottori (JP); Kazuo Azuma, Tottori (JP); Tomohiro Osaki, Tottori (JP); Shinsuke Ifuku, Tottori (JP); Hiroyuki Saimoto, Tottori (JP)

(72) Inventors: Saburo Minami, Tottori (JP); Kazuo Azuma, Tottori (JP); Tomohiro Osaki, Tottori (JP); Shinsuke Ifuku, Tottori (JP); Hiroyuki Saimoto, Tottori (JP)

(73) Assignees: National University Corporation Tottori University, Tottori (JP); Tottori Prefectural Government, Tottori (JP)

(21) Appl. No.: 14/344,962

(22) PCT Filed: Jun. 27, 2012
(86) PCT No.: PCT/JP2012/066394

§ 371 Date: Mar. 14, 2014

(30) Foreign Application Priority Data
May 15, 2011 (JP) 2011-202193

(51) Int. Cl. A61K 31/722

(52) U.S. Cl. CPC A61K 31/722
USPC 428/401; 536/20

(57) ABSTRACT

The present invention provides a therapeutic and/or prophylactic agent for inflammatory bowel disease and an NF-kB activation inhibitor, each of which comprises chitin nanofibers or chitosan nanofibers.

### TECHNICAL FIELD

The present invention relates to an agent for the treatment and/or prophylaxis of inflammatory bowel disease as well as an NF-kB activation inhibitor, each of which comprises chitin nanofibers or chitosan nanofibers.

### BACKGROUND ART

Inflammatory bowel disease (IBD) is a generic name of diseases which cause chronic inflammation in a digestive tract, and includes ulcerative colitis and Crohn's disease. In recent years, in our country, the number of patients is rapidly increasing with westernization of a diet. The etiology thereof is unknown, and it is thought that the diseases are developed by allowing some factors to act together, thereby to influence on the immune function, generating cytokine abnormality, and finally, causing abnormality in mucosa immunity.

In addition, IBD is an embarrassing disease which easily becomes chronic and easily relapses. For treating IBD, an immunosuppressant such as an adrenocortical hormone is exclusively used, however the immunosuppressant has become problematic in that the side effect is serious, and long term administration is also difficult.

Up to now, an attempt to treat IBD without using an immunosuppressant has been tried. For example, as an example of treating IBD using saccharides, a treatment agent for IBD containing N-acetylglucosamine (Patent Document 1) or a glucosamine salt (Patent Document 2) has been published, however development of a treatment agent is desired which has a further effect, has no fear of side effect, and can be administered for a long term.

### PRIOR ART DOCUMENT

### Patent Documents

[Patent Document 1] JP-B-6-78235
[Patent Document 2] JP-A-2007-291011

### SUMMARY OF THE INVENTION

### Problems to be Solved by the Invention

An object of the present invention is to provide an agent for the treatment and/or prophylaxis of IBD, which has no fear of side effects, can be administered for a long term, and has excellent effects.

### Means for Solving the Problems

In order to solve the aforementioned problems, the present inventors have intensively study, and found that when chitin nanofibers or chitosan nanofibers are administered to an animal affected with IBD, the symptom of IBD is remarkably improved, resulting in completion of the present invention.

That is, the present invention provides the followings:

(1) an agent for the treatment and/or prophylaxis of inflammatory bowel disease, comprising chitin nanofibers;

(2) the agent according to (1), wherein the chitin nanofibers are produced by the following method:

a method comprising subjecting a material derived from a chitin-containing organism to at least one deproteinization step and at least one deashing step, and then, subjecting the material to a fiber-loosening step;

(3) the agent according to (1) or (2), wherein the fiber has a width of 2 nm to 20 nm; (4) the agent according to any one of (1) to (3), which is an oral administration agent;

(5) an agent for the treatment and/or prophylaxis of inflammatory bowel disease, comprising chitosan nanofibers;

(6) the agent according to (5), wherein the chitosan nanofibers are produced by the following method:

a method comprising subjecting a material derived from a chitosan-containing organism to at least one deproteinization step and at least one deashing step and at least one deacetylation step, and then, subjecting the material to a fiber-loosening step;

(7) the agent according to (5) or (6), wherein the fiber has a width of 2 nm to 40 nm;

(8) the agent according to any one of (5) to (7), which is an oral administration agent;

(9) an agent for the inhibition of NF-kB activation, comprising chitin nanofibers; and

(10) an agent for the inhibition of NF-kB activation, comprising chitosan nanofibers.

### Effects of the Invention

According to the present invention, there are provided an agent for the treatment and/or prophylaxis of IBD, which has no fear of side effects, can be administered for a long term, and has excellent effects, as well as an agent for the inhibition of NF-kB activation.

### BRIEF DESCRIPTION OF THE DRAWINGS

FIG. 1 is a tissue section photograph showing the effect of chitin nanofibers on histological change in the large intestine of a DSS-induced ulcerative colitis mouse. On 6th day from test initiation, a histological section was obtained from each one mouse of each group of a chitin powder (+) group (upper panel), a chitin nanofiber (+) group (middle panel) and a control (+) group (lower panel). The large intestine was fixed, and a tissue section was stained with hematoxylin-eosin. Erosion is shown with an arrow. A scale bar is 100 μm.

FIG. 2 is a bar graph showing the effect of chitin nanofibers on a histological score in the large intestine of a DSS-induced ulcerative colitis mouse. The white bar is a control (+) group, the black bar is a chitin nanofiber (+) group, and the gray bar is a chitin powder (+) group. (−) is a group to which dextran sulfate sodium (DSS) was not administered. ** shows p<0.01.

FIG. 3 is a tissue section photograph showing the effect of chitin nanofibers on the MPO-positive large intestine cell number of a DSS-induced ulcerative colitis mouse. On 6th day from test initiation, concerning each group of a chitin powder (+) group (upper panel), a chitin nanofiber (+) group (middle panel) and a control (+) group (lower panel), one mouse was selected from three mice, and a tissue section was obtained. An MPO-positive cell is shown with an arrow. A scale bar is 100 μm.

FIG. 4 is a bar graph showing the effect of chitin nanofibers on the MPO-positive large intestine cell number of a DSS-induced ulcerative colitis mouse. (−) is a group to which dextran sulfate sodium (DSS) was not administered. * shows p<0.05, and ** shows p<0.01.

FIG. 5 shows the results of investigation of influence of chitin nanofibers on NF-kB activation in the large intestine epithelium of a DSS-induced ulcerative colitis mouse. FIG. 5A (a) is a microscopic image showing an NF-kB-positive region of a control (+) group (arrow), FIG. 5A (b) is a microscopic image showing an NF-kB-positive region of a chitin nanofiber (+) group (arrow), and FIG. 5A (c) is a microscopic image showing an NF-kB-positive region of a chitin powder (+) group (arrow). A bar is 100 micron. FIG. 5B is a graph showing the ratio of an NF-kB-positive area of a control (−) group, a control (+), a chitin nanofiber (+) group, and a chitin powder (+) group. * p<0.05, **: p<0.01.

### MODE FOR CARRYING OUT THE INVENTION

The present invention provides an agent for the treatment and/or prophylaxis of inflammatory bowel disease (sometimes referred to as “IBD”), comprising chitin nanofibers or chitosan nanofibers. A disease to be targeted by the agent for the treatment and/or prophylaxis of the present invention (sometimes referred to as “agent of the present invention”) is IBD. IBD includes two main diseases, ulcerative colitis and Crohn's disease. The agent of the present invention can be used for the treatment and/or prophylaxis of these diseases. The agent of the present invention can be preferably used for the treatment and/or prophylaxis of ulcerative colitis. In the present specification, prevention of relapse of IBD is also included in treatment and/or prophylaxis. In the present specification, “treatment”, “prophylaxis” and “prevention of relapse” have the meanings which are usually recognized in the medical field.

The chitin nanofibers used in the agent of the present invention have a length of the fiber is 1 μm or more. The average deacetylation degree is 5% or less, and the width (or diameter) of the fiber is relatively even, and the width (or diameter) is usually about 2 nm to about 200 nm, preferably about 2 nm to about 100 nm, more preferably about 2 nm to about 50 nm, for example, about 5 nm to about 20 nm. Preferably, the fiber is composed of an extended-chain crystalline structure. In the specification, for example, “the width (or diameter) of the chitin nanofibers is about 2 nm to about 20 nm” refers to a state where, when observed with an electron microscope, about 50% or more, preferably about 60% or more, further preferably about 70% or more of the entire fibers have a width (or diameter) of about 2 nm to about 20 nm or less. Further, the same shall apply to the width (or diameter) of chitosan nanofibers described later.

The chitosan nanofibers used in a cosmetic, bath additive and pharmaceutical composition of the present invention, have a length of the nanofiber is 1 μm or more. The average deacetylation degree of the whole nanofiber is 5% or more, and the width (or diameter) of the fiber is relatively even, and the width (or diameter) is usually about 2 nm to about 200 nm, preferably about 2 nm to about 100 nm, more preferably about 2 nm to about 50 nm, for example, about 5 nm to about 20 nm. Preferably, the fiber is composed of extended-chain crystalline structure. In such chitosan nanofibers, only a surface of an N-acetyl group of the chitin nanofiber may be selectively deacetylated.

The chitin nanofibers used in the agent of the present invention may be chitin nanofibers produced by any method/means. Chitin nanofibers are preferable which are produced by a method comprising subjecting a material derived from a chitin-containing organism to at least one deproteinization step and at least one deashing step, and then, subjecting the material to a fiber-loosening step. The aforementioned preferable method for producing chitin nanofibers and chitin nanofibers obtained by the production method will be described below (see also the specification of International Publication WO 2010/073758, the content of which is incorporated herein by reference).

The chitin nanofibers of the present invention can be obtained from a natural environment, for example, from a material derived from a chitin-containing organism. Examples of the chitin-containing organism include, but are not limited to, crustaceans such as shrimps and crabs, insects, krills and the like. Preferably, the chitin nanofibers of the present invention may be obtained from shells and teguments of an organism having a large chitin content, for example, crustaceans such as shrimps and crabs. However, chitin nanofibers in the living body cannot be obtained unless subject to a matrix removing treatment since chitin nanofibers contain a matrix containing protein and calcium carbonate, which exists in the periphery and gap. The chitin which is subjected to nanofiberization by the step described above may be chitin having an a type crystal structure such as chitin derived from a crab shell or a shrimp shell, or may be chitin having a P type crystal structure such as chitin derived from cuttlebone.

By deproteinization, the protein which surrounds chitin nanofibers to form a matrix is removed. As a deproteinization treatment, an alkali treatment method, a method using a proteolytic enzyme such as protease, and the like are exemplified, and the alkali treatment method is preferable. In the deproteinization by the alkali treatment, an aqueous solution of an alkali such as potassium hydroxide, sodium hydroxide, or lithium hydroxide is preferably used, and the concentration thereof can be appropriately selected depending on the amount of a material derived from a chitin-containing organism, the kind of a chitin-containing organism, the site and the like, and is usually about 2 to about 10% (w/v), preferably about 3 to about 7% (w/v), for example, about 5% (w/v). The temperature for deproteinization by the alkali treatment can be appropriately selected depending on the amount of a material derived from a chitin-containing organism, the kind of a chitin-containing organism, the site and the like, and is usually about 80° C. or higher, preferably about 90° C. or higher, and further preferably, the treatment is performed while the alkali aqueous solution is refluxed. The treatment time can be also appropriately selected depending on the amount of a material derived from a chitin-containing organism, the kind of a chitin-containing organism, the site and the like, and the treatment may be performed usually for several hours to about 3 days, preferably several hours to about 2 days.

By deashing, an ash component, mainly, calcium carbonate which surrounds chitin nanofibers is removed. As a deashing treatment, an acid treatment method, an ethylenediaminetetraacetic acid treatment method, and the like are exemplified, and the acid treatment method is preferable. In deashing by acid treatment, an aqueous solution of an acid such as hydrochloric acid is preferably used, and the concentration thereof can be appropriately selected depending on the amount of a material derived from a chitin-containing organism, the kind of a chitin-containing organism, the site and the like, and is usually about 4 to about 12% (w/v), preferably about 5 to about 10% (w/v). The temperature for deproteinization by acid treatment can be appropriately selected depending on the amount of a material derived from a chitin-containing organism, the kind of a chitin-containing organism, the site and the like, and is usually about 10 to about 50° C., preferably about 20 to about 30° C., and may be, for example, room temperature. The deashing time by acid treatment can be also appropriately selected the amount of a material derived from a chitin-containing organism, the kind of a chitin-containing organism, the site and the like, and the deashing may be performed usually for several hours to several days, preferably about 1 to about 3 days, and for example, 2 days.

Then, the tegument (almost all of which is converted into chitin nanofibers) obtained in the aforementioned step is subjected to a fiber-loosening treatment to obtain the intended chitin nanofibers. Since chitin nanofibers undergo hydrogen bonding and strongly aggregate when dried, it is preferable that each step of the method for producing the chitin nanofibers of the present invention is always performed without drying the materials. In the fiber-loosening treatment with the addition of an acid, devices such as a stone mill type triturating machine, a high-pressure homogenizer and a freezed crushing machine can be used, and a grinder treatment is preferably performed by the stone mill type triturating machine. When a device capable of applying a stronger load such as the stone mill type triturating machine is used, even alpha-chitin derived from shells of crab and shrimp can be quickly fiber-loosened.

In the method for producing the chitin nanofibers, if necessary or if desired, a decoloration step may be performed. The decoloration step may be performed at any stage of the method described above, and is preferably performed after completion of the deproteinization and deashing treatment. The decoloration may be performed by any method, and it is preferred to be subjected to extraction with an organic solvent such as an alcohol, or to use a chlorine-based bleaching agent, an oxygen-based bleaching agent or a reducing bleaching agent. The decoloration may be performed in a buffer solution, for example, an acetate buffer solution at about 70 to about 90° C. for several hours using about 1 to about 2% sodium hypochlorite.

Furthermore, a crushing step may be performed so as to efficiently perform the deproteinization step, the deashing treatment step, the decoloration step, the fiber-loosening step, and a treatment with an acidic reagent described hereinafter. The crushing step may be performed at any stage of the method described above, and is preferably performed immediately before the fiber-loosening step. Although the crushing step may be performed by any method, a homogenizer treatment method and a mixer treatment method are preferable and, for example, the crushing step may be performed by a domestic food processor.

The aforementioned steps such as deproteinization step, deashing treatment step, decoloration step and crushing step may be performed repeatedly, plural times, or alternately. Each step may be performed in any order.

Further, if necessary or if desired, water dispersibility of the chitin nanofibers may be improved by treating the deashed chitin-containing material with an acidic reagent. Since the chitin nanofibers obtained by the fiber-loosening step are thin and uniform by performing the treatment with an acidic reagent, the water dispersibility of the chitin nanofibers is improved. When the fibers are fine and the water dispersibility is improved, a coating film which is formed when applied on the skin is uniform and an advantageous effect such as a moisturizing effect is exerted. Since the acidic reagent generates a positive charge on a chitin fiber surface, this is advantageous for effectively loosening firmly aggregated chitin fibers. Therefore, by using the acidic reagent, it is also possible to easily loosen a chitin aggregate obtained by drying after the deproteinization step, the deashing treatment step and the decoloration step. There is no particular limitation on the treating method using the acidic reagent, and the method may be a method of impregnating the material with the acidic reagent. The treatment with the acidic reagent can be typically performed by immersing the deashed chitin-containing material in an aqueous solution of an acid. In this step, not only the water dispersibility can be improved, but also unevenness in a fiber width (or fiber diameter) of the chitin nanofibers can be suppressed. The acid which can be used in this step may be any acid and is not particularly limited, and is preferably a weak acid. Examples of the weak acid include, but are not limited to, acetic acid, formic acid, chloroacetic acid, fluoroacetic acid, propionic acid, butyric acid, lactic acid, citric acid, malonic acid, ascorbic acid and the like. The weak acid used in this step is preferably acetic acid. In this step, the pH of an aqueous solution of the weak acid is usually adjusted to about 2 to about 5, preferably about 2.5 to about 4.5, for example, about 3 to about 4. The temperature of this step can be appropriately selected depending on the amount of a material derived from a chitin-containing organism, the kind of a chitin-containing organism, the site and the like, and may be usually about 10 to about 50° C., preferably about 20 to about 30° C., for example, room temperature. The treatment time of this step can be also appropriately selected depending on the amount of a material derived from a chitin-containing organism, the kind of a chitin-containing organism, the site and the like, and may be usually for 1 hour to about 1 day, and preferably for about 3 to about 12 hours, for example, overnight. The treatment step using an acid may be performed at any stage before the fiber-loosening step, and may be preferably performed at a stage where purification of the chitin nanofibers has proceeded to some degree after the deproteinization and deashing and, for example, the treatment step may be performed immediately before the fiber-loosening step.

The chitin nanofibers which can be obtained by the aforementioned production method are thin and uniform, and are extremely long, and are composed of an extended-chain crystalline structure. The width (or diameter) of the chitin nanofibers obtained by the aforementioned production method is relatively even, and the width (or diameter) is usually about 2 nm to about 200 nm, preferably about 2 nm to about 100 nm, more preferably about 2 nm to about 50 nm, for example, about 5 nm to about 20 nm.

Further, in the agent of the present invention, chitosan nanofibers can be also used in place of the chitin nanofibers, or by mixing with the chitin nanofibers. The chitosan nanofibers are a deacetylated product of the chitin nanofibers, and may be partially deacetylated, or may be completely deacetylated.

The chitosan nanofibers used in the agent of the present invention may be chitosan nanofibers produced by any method/means, and chitosan nanofibers produced by subjecting a material derived from a chitin-containing organism to at least one deproteinization step and at least one deashing step and at least one deacetylation step, and then, subjecting the material to a fiber-loosening step are preferable. The preferable method for producing chitosan nanofibers and chitosan nanofibers obtained by the production method will be described below (see also the specification of International Publication WO 2010/073758).

The chitin-containing material, the deproteinization step, the deashing step and the fiber-loosening step are the same as those described above regarding the production of the chitin nanofibers. In addition, in the present invention, the deproteinization step and the deacetylation step can be also performed simultaneously. Further, by subjecting a commercially available chitin powder which has been already subjected to the deproteinization step and the deashing step, to the deacetylation step, the chitosan nanofibers can be also produced.

As for an acetylation method, several methods are known, and an alkali treatment method is preferable. In the deacetylation by the alkali treatment, an aqueous solution of an alkali such as potassium hydroxide, sodium hydroxide, or lithium hydroxide is preferably used, and the concentration thereof is usually about 20 to about 50% (w/v), preferably about 30 to about 40% (w/v), for example, about 40% (w/v). The temperature for deacetylation by the alkali treatment can be appropriately selected depending on the amount of a material derived from a chitin-containing organism, the kind of a chitin-containing organism, the site and the like, and is usually about 80° C. or higher, preferably about 90° C. or higher, and further preferably, the treatment is performed while the alkali aqueous solution is refluxed. The treatment time can be also appropriately selected depending on the amount of a material derived from a chitin-containing organism, the kind of a chitin-containing organism, the site and the like, and the treatment may be performed for usually 30 minutes to about 3 days, preferably, 30 minutes to overnight. In addition, in order to avoid aggregation of the chitosan nanofibers, it is very preferable that each step of the method for producing the chitosan nanofibers of the preset invention is always performed without drying materials.

The width (or diameter) of the chitosan nanofibers produced by the aforementioned production method is relatively even, and the width (or diameter) is usually about 2 nm to about 200 nm, preferably about 2 nm to about 100 nm, more preferably about 2 nm to about 50 nm, for example, about 5 nm to about 20 nm. The fiber state is an extended-chain crystalline structure.

The chitin nanofibers or chitosan nanofibers used in the agent of the present invention may be modified or derivatized, or may be in the form of a salt (e.g., hydrochloride, etc.). For example, hydrogen of a hydroxy group at 3-position of a sugar, a hydroxy group at 6-position of a sugar, or a hydroxy group at an end of a sugar chain may be substituted with other groups such as an alkyl group. Alternatively, a methyl group in an acetyl group at 2-position of a sugar of chitin may be substituted with other groups such as an ethyl group. Alternatively, hydrogen of an amino group at 2-position of a sugar of chitosan may be substituted with other groups such as an alkyl group, or may form a salt with an anion such as a halide ion. These modifications and derivatives or salts are absolutely exemplification, and are not limitative. In the present specification, these modifications and derivatives or salts are also included in the chitin nanofibers or the chitosan nanofibers. Such derivatives and modifications or salts are known to a person skilled in the art, and a method for producing them is also known.

The chitin nanofibers or the chitosan nanofibers are a natural product contained in crabs, shrimps and so on, and are very high in safety. Therefore, the agent of the present invention comprising the chitin nanofibers or the chitosan nanofibers can be administered over a long period of time, and has no side effect, or if any, extremely small side effect. Since the agent of the present invention is suitable for long term administration, it is extremely effective for inflammatory bowel disease which easily becomes chronic and easily relapses. On the other hand, immunosuppressants such as adrenocortical hormones are exclusively used for inflammatory bowel disease, however these agents are serious in side effect, and long term administration is also difficult. Therefore, the agent of the present invention solves the problems of the immunosuppressant which has previously been used for treating inflammatory bowel disease. Moreover, as shown in Examples, the agent of the present invention is also excellent in terms of the effect of the treatment and/or prophylaxis of inflammatory bowel disease.

The agent of the present invention can be formulated into known various dosage forms. As a dosage form of the agent of the present invention, an oral agent is particularly preferable. Examples of the oral agent include, but are not limited to, tablets, granules, powders, capsules, drinks and the like. These dosage forms can be produced according to a known method. The agent of the present invention may be in the form of food or supplement, in addition to the form of a pharmaceutical composition.

In a further aspect, the present invention provides a method for the treatment and/or prophylaxis of inflammatory colitis, comprising administering an effective amount of chitin nanofibers to a subject in need of the treatment and/or prophylaxis of inflammatory colitis.

In a further aspect, the present invention provides use of chitin nanofibers for the production of a medicament for the treatment and/or prophylaxis of inflammatory colitis. In a further aspect, the present invention relates to use of chitin nanofibers for the treatment and/or prophylaxis of inflammatory colitis.

The chitin nanofibers to be preferably used for the method or use above are produced by the following method:

a method comprising subjecting a material derived from a chitin-containing organism to at least one deproteinization step and at least one deashing step, and then, subjecting the material to a fiber-loosening step.

In a further aspect, the present invention provides a method for the treatment and/or prophylaxis of inflammatory colitis, comprising administering an effective amount of chitosan nanofibers to a subject in need of the treatment and/or prophylaxis of inflammatory colitis.

In a further aspect, the present invention provides use of chitosan nanofibers for the production of a medicament for the treatment and/or prophylaxis of inflammatory colitis. In a further aspect, the present invention relates to use of chitosan nanofibers for the treatment and/or prophylaxis of inflammatory colitis.

The chitosan nanofibers to be preferably used for the method or use above are produced by the following method:

a method comprising subjecting a material derived from a chitosan-containing organism to at least one deproteinization step and at least one deashing step and at least one deacetylation step, and then, subjecting the material to a fiber-loosening step.

Usually, the dose of the chitin nanofibers or the chitosan nanofibers which are administered for the treatment and/or prophylaxis of inflammatory bowel disease can be appropriately determined by a physician while the physician observes the effects. Generally, the dose of the chitin nanofibers or the chitosan nanofibers per adult per day is usually about 1 g to about 50 g, preferably about 1 g to about 5 g, but the dose is not limited thereto, and can be appropriately changed depending on the severity of the symptom, the weight and health state of a patient, and the like.

According to research by the present inventors, the effect of the chitin nanofibers for inhibiting inflammatory colitis was found to be obtained by inhibiting the activation of NF-kB. It is thought that there is also a similar effect regarding the chitosan nanofibers. Therefore, in a further aspect, the present invention provides the following aspects.

(i) An NF-kB activation inhibitor comprising chitin nanofibers.

(ii) Use of chitin nanofibers for the production of a medicament which inhibits the activity of NF-kB.

(iii) Use of chitin nanofibers for inhibiting the activity of NF-kB.

(iv) An agent for the inhibition of NF-kB activation, comprising chitosan nanofibers.

(v) Use of chitosan nanofibers for the production of a medicament which inhibits the activity of NF-kB.

(vi) Use of chitosan nanofibers for inhibiting the activity of NF-kB.

The chitin nanofibers to be preferably used for the agent or use above are preferably produced by the following method:

a method comprising subjecting a material derived from a chitin-containing organism to at least one deproteinization step and at least one deashing step, and then, subjecting the material to a fiber-loosening step.

The chitosan nanofibers to be preferably used for the agent or use above are produced by the following method:

a method comprising subjecting a material derived from a chitosan-containing organism to at least one deproteinization step and at least one deashing step and at least one deacetylation step, and then, subjecting the material to a fiber-loosening step.

Usually, the dose of the chitin nanofibers or the chitosan nanofibers which are administered to a subject for inhibiting the activity of NF-kB can be appropriately determined by a physician while the physician observes the effects. Generally, the dose of the chitin nanofibers or the chitosan nanofibers per adult per day is usually about 1 g to about 50 g, preferably about 1 g to about 5 g, but the dose is not limited thereto, and can be appropriately changed depending on the severity of the symptom, the weight and health state of a patient, and the like.

### EXAMPLES

The present invention will be described in more detail and specifically below by way of examples, but the examples are illustrative description, and do not limit the present invention.

### Example 1

### I. Experimental Method and Material

### (1) Reagents

Chitin nanofibers were prepared from a crab shell of red snow crab by the method described in the specification of International Publication WO 2010/073758. Chitin powders were purchased from Nacalai Tesque (Lot No.: M0A3811; Kyoto, Japan). The chitin powders had an average particle diameter of about 200 micron. Dextran sulfate sodium (DSS) (molecular weight 36 to 50 kDa; reagent grade) was purchased from MP Biochemicals LLC (Solon, Ohio, USA).

### (2) Animal

A C57BL/6 mouse (female, 6 week old) was purchased from CLEA Japan (Osaka, Japan).

### (3) An Experimental Animal Group and an Administration Method

Mice (n=68) were randomly assigned to the following six groups:

Control (+) group (n=17): Only DSS* was administered to this group.

Control (−) group (n=5): Tap water was administered to this group.

Chitin nanofiber (+) group (n=17): Chitin nanofibers** and DSS* were administered to this group.

Chitin nanofiber (−) group (n=7): Only chitin nanofibers** were administered to this group.

Chitin powder (+) group (n=16): Chitin powders*** and DSS* were administered to this group.

Chitin powder (−) group (n=16): Only chitin powders*** were administered to this group.

DSS which was dissolved in drinking water to have a concentration of 3%. Fed ad libitum.

** Chitin nanofibers were suspended in water which had been regulated with acetic acid at a pH of 3 to have a concentration of 1%, so that a gel was obtained, and the resulting gel-like substance was dissolved in drinking water to have a concentration of 0.1%, and this was fed ad libitum.

*** Chitin powders were suspended in water which had been regulated with acetic acid at a pH of 3 to have a concentration of 1%, and the resulting suspension was dissolved in drinking water to have a concentration of 0.1%, and this was fed ad libitum.

In the control (+) group, the chitin nanofiber (+) group and the chitin powder (+) group, for a term from the test initiation day (0th day) to 6 days after test initiation, DSS was administered to induce colitis. In the chitin nanofiber (+) group and the chitin nanofiber (−) group, chitin nanofibers were administered over 7 days before test initiation (the chitin nanofiber gel was dissolved in drinking water to have a concentration of 0.1%, and this was fed ad libitum). In the chitin powder (+) group and the chitin powder (−) group, a chitin powder was administered over 7 days before test initiation (the chitin powder suspension was dissolved in drinking water to have a concentration of 0.1%, and this was fed ad libitum). A common feed was given to each group.

On 3rd day and 5th day from test initiation, blood was collected and a large intestine sample was collected from animals of the control (+) group, the chitin nanofiber (+) group and the chitin powder (+) group (each group n=5). On 6th day from test initiation, blood was collected and a large intestine sample was collected from animals of all groups (each group n=5 to 7).

### (4) Clinical Analysis

Ulcerative colitis of an animal was evaluated using a disease activity index (DAI). DAI was evaluated in accordance with the method described in Melger et al., American Journal of Physiology Gastrointestinal and Liver Physiology, 288 (6), G1328-1338. An example of DAI is shown in Table 1.

TABLE 1

| | Weight loss | | |
|---|---|---|---|
| | (Decrease % relative | | |
| Score | to initial weight) | Diarrhea | Bloody stool |
| 0 | <5% | Normal | Normal |
| 1 | 5-10% | Slight loose | Slight bloody |
| | | stool | stool |
| 2 | 10-20% | Loose stool | Bloody stool |
| 3 | >20% | Water-like | Bleeding from |
| | | stool | anus all times |

The length and weight of the large intestine were also measured. A large intestine tissue was treated and processed in order to use the tissue in histological evaluation described below.

### (5) Histological Evaluation of Colitis

The large intestine tissue was fixed in 10% buffered formalin. A section (thickness 3 micron) was made from each sample, and stained with hematoxylin-eosin, and then histological observation was performed. Each resulting section was observed with a microscope, and histological score was performed. The histological score was performed as described in Ohkawara et al. Scandinavian Journal of Gastroenterology, 40 (9), 1049-1057 (2005).

Score 0: normal mucosa;

Score 1: infiltration of inflammatory cell;

Score 2: the length of intestinal villi is not shortened to half of the normal length (shortening of the crypt by less than half of the height);

Score 3: the length of intestinal villi is shortened to half or more of the normal length (shortening of the crypt by more than half of the height);

Score 4: intestinal villi are lost (crypt loss);

Score 5: destruction of epithelial cell. Histological score was performed in 10 fields at a magnification of 100 using three mice per each group. The average of the total of 30 fields was adopted as a score of each group.

### (6) Myeloperoxidase (MPO) Staining

MPO staining is a marker of entry of a lymphocyte into a tissue. The MPO staining was performed in accordance with the method described in Schneidhelm et al. Clinical Chemistry, 55 (8), 1462-1470 (2009). MPO-positive cells in a submucosal tissue layer were counted in 20 fields at a magnification of 400 using three mice per each group. The total of 60 fields was adopted as the MPO-positive cell number of each group.

### (7) Measurement of Serum IL-6 Concentration

The serum IL-6 concentration was measured using a Mouse IL-6 ELISA kit (Thermo SCIENTIFIC, Rockford, Ill., USA).

### (8) Statistical Analysis

Data was expressed as average value±standard deviation. The statistical analysis was performed using 1-way ANOVA and Tukey-Kramer' test. In the case where a p value is smaller than 0.05, this was regarded as statistically significant.

### II. Result

### (1) Effect of Chitin Nanofibers on DAI of DSS-Induced Ulcerative Colitis Mouse

In the control (+) group and the chitin powder (+) group on 3rd day from test initiation, and in the chitin nanofiber (+) group on 4th day from test initiation, weight loss and loose stoop and bleeding from anus were observed (Table 2). On 4th to 6th days from test initiation, DAI was significantly lower (p<0.05) in the chitin nanofiber (+) group than in the control (+) group, and on 5th day and 6th day from test initiation, DAI was significantly lower (p<0.01, p<0.05, respectively) in the chitin nanofiber (+) group than in the chitin powder (+) group (Table 2). In the control (−) group, the chitin nanofiber (−) group and the chitin powder (−) group, a change in DAI was not observed (data are not shown).

TABLE 2

| | 0th day | 1st day | 2nd day | 3rd day | 4th day | 5th day | 6th day |
|---|---|---|---|---|---|---|---|
| Control (+) | 0.0 ± 0.0 | 0.0 ± 0.0 | 0.0 ± 0.0 | 0.3 ± 0.2 | 1.1 ± 0.4 | 3.6 ± 0.3 | 6.9 ± 0.5 |
| group | | | | | | | |
| Chitin | 0.0 ± 0.0 | 0.0 ± 0.0 | 0.0 ± 0.0 | 0.0 ± 0.0 | 0.2 ± 0.1* | 2.3 ± 0.3*^{††} | 5.1 ± 0.4*† |
| nanofiber (+) | | | | | | | |
| group | | | | | | | |
| Chitin | 0.0 ± 0.0 | 0.0 ± 0.0 | 0.0 ± 0.0 | 0.3 ± 0.2 | 1.0 ± 0.4 | 4.0 ± 0.5 | 7.0 ± 0.8 |
| powder (+) | | | | | | | |
| group | | | | | | | |

*When the chitin nanofiber (+) group and the chitin powder (+) group were compared, p < 0.05.
†When the chitin nanofiber (+) group and the chitin powder (+) group were compared, p < 0.05.
^{††}When the chitin nanofiber (+) group and the chitin powder (+) group were compared, p < 0.01.

### (2) Effect of Chitin Nanofibers on the Large Intestine Length and the Ratio of Large Intestine Weight/Length (Hypertrophy of Large Intestine Wall) of a DSS-Induced Ulcerative Colitis Mouse

It is known that the large intestine of a mouse is shortened, and the large intestine wall is thickened, by administration of DSS (Melger et al. American Journal of Physiology Gastrointestinal and Liver Physiology, 288′6), G1328-1338 (2005)). On 3rd, 5th and 6th days from test initiation, the large intestine was significantly lengthened in the chitin nanofiber (+) group as compared with the control (+) group (On 3rd day and 5th day, p<0.05; and on 6th day, p<0.01) (Table 3 (a)). Further, on 3rd, 5th and 6th days from test initiation, the large intestine was significantly lengthened in the chitin nanofiber (+) group as compared with the chitin powder (+) group (On 3rd and 5th days, p<0.05; and on 6th day, p<0.01) (Table 3 (a)). In the control (−) group, the chitin nanofiber (−) group and the chitin powder (−) group, there was no change in the large intestine length (data are not shown). On 5th and 6th days from test initiation, in the chitin nanofiber (+) group, the large intestine wall was thinned as compared with the control (+) group. On 5th day from test initiation, in the chitin nanofiber (+) group and the chitin powder (+) group, the large intestine wall was significantly thinned as compared with the control (+) group (p<0.05) (Table 3 (b)). On 6th day from the test, in the chitin nanofiber (+) group, the large intestine wall was significantly thinned as compared with the control (+) group and the chitin powder (+) group (p<0.01) (Table 3 (b)). In the control (−) group, the chitin nanofiber (−) group and the chitin powder (−) group, there was no change in the thickness of the large intestine wall (data are not shown).

TABLE 3

| | | | |
|---|---|---|---|
| 0th day | 3rd day | 5th day | 6th day |

(a) Large intestine length (cm)

| | | | | |
|---|---|---|---|---|
| Control (+) | 7.4 ± 0.1 | 6.7 ± 0.2 | 5.1 ± 0.2 | 4.9 ± 0.1 |
| group | | | | |
| Chitin | 7.3 ± 0.1 | 7.5 ± 0.2*† | 7.0 ± 0.1**†† | 6.3 ± 0.1**†† |
| nanofiber (+) | | | | |
| group | | | | |
| Chitin | 7.4 ± 0.2 | 6.6 ± 0.2 | 5.7 ± 0.1 | 5.3 ± 0.2 |
| powder (+) | | | | |
| group | | | | |

(b) Ratio of large intestine weight/length (mg/cm)

| | | | | |
|---|---|---|---|---|
| Control (+) | 26.8 ± 0.8 | 31.9 ± 1.5 | 50.2 ± 3.9 | 40.8 ± 2.7 |
| group | | | | |
| Chitin | 29.8 ± 0.3 | 29.8 ± 0.5 | 38.4 ± 1.2* | 33.3 ± 1.2*† |
| nanofiber (+) | | | | |
| group | | | | |
| Chitin | 28.8 ± 1.7 | 27.2 ± 0.5 | 37.4 ± 1.4* | 41.7 ± 2.5 |
| powder (+) | | | | |
| group | | | | |

*On each measurement day, p < 0.05 as compared with the control (+) group.
**On each measurement day, p < 0.01 as compared with the control (+) group.
†On each measurement day, p < 0.05 as compared with the control (+) group.
††On each measurement day, p < 0.01 as compared with the control (+) group.

### (3) Effect of Chitin Nanofibers on Histological Change in the Large Intestine of a DSS-Induced Ulcerative Colitis Mouse

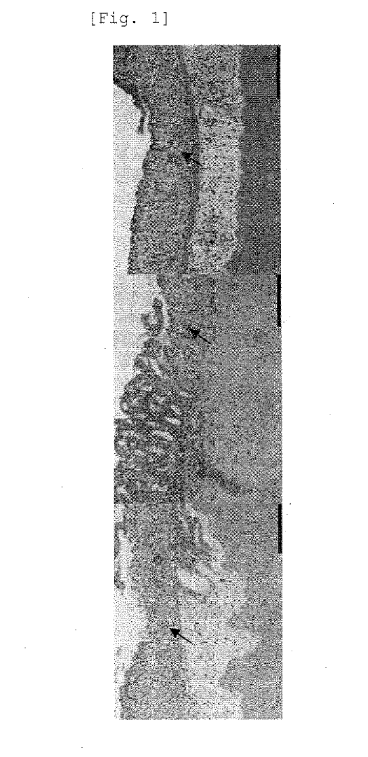

Histological grading was performed by microscopy to evaluate damage of the intestinal mucosa. In the control (−) group, the chitin nanofiber (−) group and the chitin powder (−) group, histological change was not seen. On 3rd day from test initiation, in the control (+) group, the chitin nanofiber (+) group and the chitin powder (+) group, infiltration of an inflammatory cell was observed. On 5th day from test initiation, in the control (+) group and the chitin powder (+) group, erosion, shortening or destruction of crypt, as well as edema were seen. On 5th day from test initiation, some sites of erosion were seen in the chitin nanofiber (+) group, but the shortening or destruction of crypt was remarkably inhibited, and edema was also slightly inhibited. On 6th day from test initiation, in the control (+) group and the chitin powder (+) group, severe erosion, destruction of crypt, and edema were observed, and further, some ulcers were also observed. In the chitin nanofiber (+) group, erosion, destruction of crypt and, edema were remarkably inhibited as compared with the control (+) group and the chitin powder (+) group (FIG. 1).

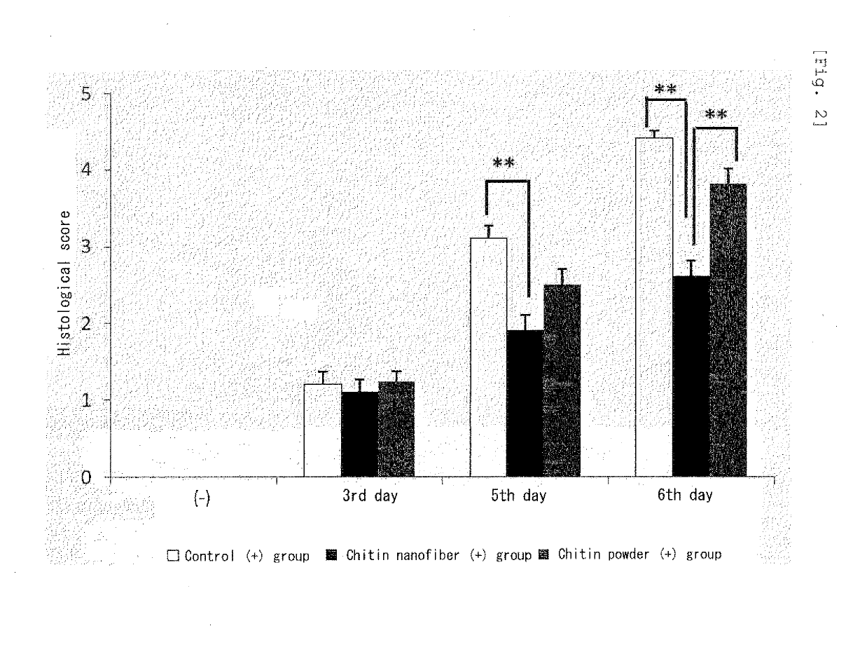

Further, severity of tissue damage was investigated by histological score using a section stained with hematoxylin-eosin. On 5th day from test initiation, in the chitin nanofiber (+) group, the histological score was significantly decreased (p<0.01) as compared with the control (+) group, and on 6th day, the histological score was significantly decreased (p<0.01) as compared with the control (+) group and the chitin powder (+) group (FIG. 2).

### (4) Effect of Chitin Nanofibers on the MPO-Positive Large Intestine Cell Number of a DSS-Induced Ulcerative Colitis Mouse

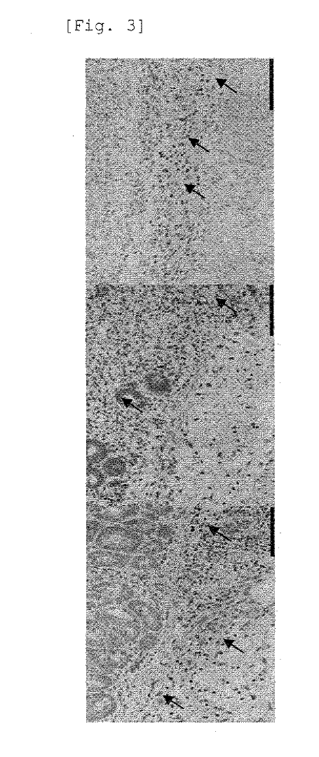

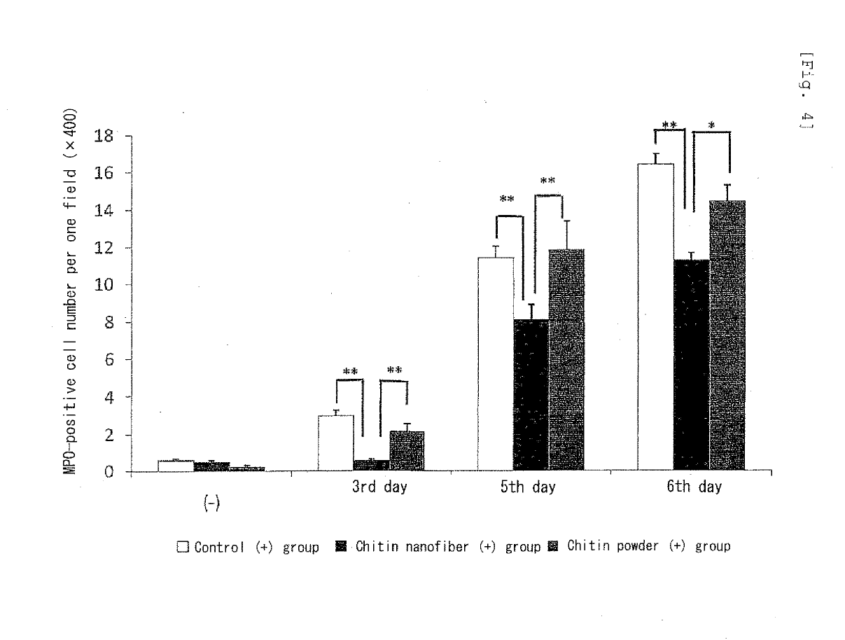

The results of MPO staining on 6th day from test initiation of each group are shown in FIG. 3, and the MPO-positive cell number is shown in FIG. 4. In the control (−) group, the chitin nanofiber (−) group and the chitin powder (−) group, 0 to 1 MPO-positive cell was observed (observation was performed at a magnification of 400; data are not shown). On 3rd day to 6th day from test initiation, in the control (+) group and the chitin nanofiber (+) group, the MPO-positive cell number was gradually increased. However, on 3rd, 5th and 6th days from test initiation, in the chitin nanofiber (+) group, the MPO-positive cell number was significantly smaller as compared with the control (+) group (p<0.01). Further, on 3rd, 5th and 6th days from test initiation, in the chitin nanofiber (+) group, the MPO-positive cell number was significantly smaller as compared with the chitin powder (+) group (On 3rd day and 5th day, p<0.01; and on 6th day, p<0.05).

### (5) Effect of Chitin Nanofibers on the Serum ID-6 Concentration of a DSS-Induced Ulcerative Colitis Mouse

It is known that IL-6 is a central cytokine in IBD, and contributes to an improvement in the T cell survival ratio and apoptosis resistance at an inflammation site (Mudeter et al. Inflammatory Bowel Disease, 13 (8), 1016-1023 (2007)). On 5th day from test initiation, in the chitin nanofiber (+) group (85.8±1.2 pg/ml), the serum IL-6 concentration was significantly lower as compared with the control (+) group (273.1±41.9 pg/ml) (p<0.01).

From the foregoing experimental results, it was confirmed that the chitin nanofibers significantly improve the symptom of IBD and damage of a large intestine tissue, but such an effect is not seen in the chitin powder. Further, in the above experiment, the chitin nanofibers did not cause a decrease in the weight of an animal, and the side effect was not also seen. Therefore, it was understood that the agent of the present invention comprising the chitin nanofibers or the chitosan nanofibers is effective in the treatment and/or prophylaxis of IBD.

### Example 2

It has been reported that a nuclear factor kB (NF-kB) plays a central role in some endogenous immune signaling pathways. It has been also reported that NF-kB is a transcription factor necessary for expressing a gene associated with proinflammatory response (Elson et al. (2005) Immunological Reviews. 206 (1), 260-276). Further, it has been reported that NF-kB is activated in the large intestine mucosa of inflammatory colitis (Reed et al. (2005) Digestive Disease and Science, 50 (12), 2366-2378; Visekruna et al. (2006) Journal of Clinical Investigation, 116 (12), 3195-3203). It has been also reported that when the symptom of active inflammatory colitis is exhibited, the NF-kB activity is increased in the large intestine (Zarubin and Han. (2005) Cell Research, 15 (1), 11-18). Then, effects of the chitin nanofibers on activation of NF-kB were investigated.

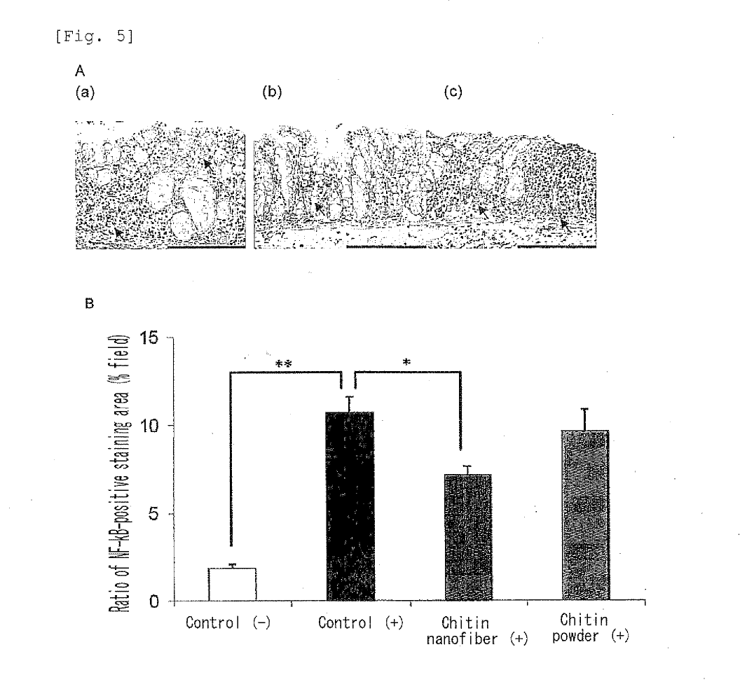

A large intestine tissue section was obtained from mice of the chitin nanofiber (+) group, the chitin powder (+) group, the control (+) group and the control (−) group. The large intestine tissue section (3 micron) on a slide glass was deparaffinized, washed with ethanol and water, and immersed in PBS. The section was microwave-treated for 5 minutes in a 0.01 M citrate buffer (pH 6.0). Thereafter, the section was washed with PBS, and incubated at room temperature for 30 minutes in 1% hydrogen peroxide methanol. The section was washed with PBS, and incubated at room temperature for 60 minutes with a rabbit polyclonal anti-NF-kB p65 antibody (1:500, sc-372; Santa Cruz Biotechnology, Inc., California, USA). The slide was washed with PBS, and colored with diaminobenzidine tetrahydrochloride. Counter staining was performed with hematoxylin. This immunohistological staining view is shown in FIG. 5A. The results of calculation of the ratio of a NF-kB-positive staining area by performing digital image analysis on 30 fields at a magnification of 100 are shown in FIG. 5B. As compared with the NF-kB-positive area in an epithelial cell of the control (+) group and the chitin powder (+) group, that of the chitin nanofiber (+) group was remarkably small. In addition, an experiment was performed also in the control (−) group, the chitin nanofiber (−) group and the chitin powder (−) group, but the ratio of the NF-kB-positive staining area was 1.8 to 3.0%.

These results indicate that the chitin nanofibers have an NF-kB activation inhibiting action, and thereby, inhibits inflammatory colitis.

### INDUSTRIAL APPLICABILITY

The present invention provides a treatment and/or prophylactic agent for inflammatory bowel disease, which contains a natural product as an active ingredient, is high in safety, and has an excellent effect, and therefore, it can be utilized in the fields of medicaments, food and the like.

1. An agent for the treatment and/or prophylaxis of inflammatory bowel disease, comprising chitin nanofibers.

2. The agent according to claim 1, wherein the chitin nanofibers are produced by the following method:
a method comprising subjecting a material derived from a chitin-containing organism to at least one deproteinization step and at least one deashing step, and then, subjecting the material to a fiber-loosening step.

3. The agent according to claim 1, wherein the fiber has a width of 2 nm to 20 nm.

4. The agent according to claim 1, which is an oral administration agent.

5. An agent for the treatment and/or prophylaxis of inflammatory bowel disease, comprising chitosan nanofibers.

6. The agent according to claim 5, wherein the chitosan nanofibers are produced by the following method:
a method comprising subjecting a material derived from a chitosan-containing organism to at least one deproteinization step and at least one deashing step and at least one deacetylation step, and then, subjecting the material to a fiber-loosening step.

7. The agent according to claim 5, wherein the fiber has a width of 2 nm to 40 nm.

8. The agent according to claim 5, which is an oral administration agent.

9. An agent for the inhibition of NF-kB activation, comprising chitin nanofibers.

10. An agent for the inhibition of NF-kB activation, comprising chitosan nanofibers.
